(19) United States
(12) Patent Application Publication
(10) Pub. No.: US 2026/0331276 A1
(43) Pub. Date: Oct. 22, 2026

(54) SYSTEM AND METHOD FOR BEHAVIORAL ACCOUNTABILITY USING PHYSIOLOGICAL DATA AND REAL-TIME USER SELF-ASSESSMENT

(71) Applicant: Dallas Rhoads, Philadelphia, PA (US)

(72) Inventor: Dallas Rhoads, Philadelphia, PA (US)

(21) Appl. No.: 19/653,343

(22) Filed: Apr. 21, 2026

Related U.S. Application Data
(60) Provisional application No. 63/792,375, filed on Apr. 22, 2025.

(51) Int. Cl. G16H 40/63

(52) U.S. Cl. CPC G16H 40/63

(57) ABSTRACT

A computer-implemented behavioral accountability system and an application executed on a mobile computing device. The application receives physiological data of a user, including at least two physiological parameters comprising Electrocardiogram (ECG/EKG), Electroencephalography (EEG), Electromyography (EMG), breathing rate, resting heart rate, present heart rate, body temperature, heart rate variability (HRV), resting heart rate, present heart rate, and other statistical or derived metrics. The application establishes a baseline physiological profile and monitors incoming data to detect deviations indicative of a change in physiological state. In response to detecting a deviation, the application generates a notification prompting the user to perform a behavioral self-assessment. The user provides input representing a behavioral state, including the physiological data, to generate behavioral records. The application further analyzes the records to generate behavioral insights and identify patterns. The application facilitates real-time self-reflection, improves behavioral awareness, and promotes accountability during user interactions.

### CROSS-REFERENCE TO RELATED APPLICATION

The present application claims priority to, and the benefit of, U.S. Provisional Application No. 63/792,375 which was filed on Apr. 22, 2025 and is incorporated herein by reference in its entirety.

### FIELD OF THE INVENTION

The present invention generally relates to behavioral monitoring systems. More specifically, the present invention relates to a smartphone-based accountability application configured to monitor physiological data of a user and facilitate real-time behavioral self-assessment and alignment. The invention comprises an accountability application executed on a mobile computing device and configured to receive physiological data, including heart rate information, from one or more sensors associated with a wearable device and/or the mobile device. The application includes functional modules for data acquisition, baseline physiological profiling, and trigger detection to identify deviations in physiological state indicative of emotional or cognitive changes. In response to such deviations, the application generates notification prompts via a user interface, enabling the user to evaluate whether their words, actions, and behaviors are aligned with their internal thoughts and emotions. In some embodiments, the application further includes an interactive interface for receiving user input corresponding to behavioral state and justification, and a data analysis component configured to correlate physiological data with user responses to generate behavioral insights and patterns. Accordingly, the present disclosure makes specific reference thereto. Nonetheless, it is to be appreciated that aspects of the present invention are also equally applicable to other like applications, devices, and methods of manufacture.

### BACKGROUND

By way of background, during communication, in many situations, individuals may lack sufficient awareness of the relationship between their internal cognitive and/or emotional state and their external words, actions, and behaviors. As a result, individuals may fail to take ownership of their statements or conduct, which can lead to misunderstandings, disagreements, and interpersonal conflict.

In particular, during conversations or interactions with others, individuals may exhibit vagueness, inconsistency, or lack of clarity in expressing their thoughts. Such lack of clarity may reduce transparency and contribute to misinterpretation by others. Additionally, individuals may experience cognitive biases, emotional reactivity, or tunnel vision, which can impair logical reasoning and hinder effective communication. Such factors may prevent individuals from accurately evaluating their own behavior or recognizing them when their actions are misaligned with their intentions or emotions.

Conventional communication tools and applications generally do not provide mechanisms for real-time self-assessment or behavioral accountability. Furthermore, existing systems typically do not utilize physiological data to assist individuals in recognizing changes in emotional or cognitive state that may influence behavior. Accordingly, individuals desire an improved system and method that enable individuals to monitor their internal state, evaluate their behavior in real time, and promote accountability and alignment during interactions with others.

Therefore, there exists a long-felt need in the art for an improved system and method for promoting behavioral accountability during interpersonal interactions. There is a need for a solution that enables individuals to take ownership of their words, actions, and behaviors in real time, particularly during conversations that can involve emotional or cognitive stress. Additionally, there exists a need for a system that reduces ambiguity and vagueness in communication, thereby improving transparency and minimizing misunderstandings. Moreover, there is a need for a mechanism that assists individuals in recognizing cognitive biases, emotional reactivity, and illogical thinking patterns, including tunnel vision, that may impair effective communication. Finally, there is a need for a system that provides timely self-assessment prompts and encourages alignment between an individual's internal state and externally expressed behavior.

The subject matter disclosed and claimed herein, in one embodiment, comprises a computer-implemented behavioral accountability system implemented as a smartphone application configured to monitor physiological data of a user and facilitate behavioral self-assessment. The system includes an accountability software application executed on a mobile computing device and configured to receive physiological data, including heart rate information, from one or more sensors associated with a wearable device and/or the mobile computing device. The application is further configured to establish a baseline physiological profile of the user and to detect deviations indicative of changes in physiological and/or emotional state. In response to detecting such deviations, the application generates a notification prompting the user to evaluate their current behavior and determine whether their words, actions, and behaviors are aligned with their internal thoughts, feelings, and emotions.

In one embodiment, the behavioral accountability system further includes an interactive user interface configured to receive user input corresponding to a behavioral state, including classifications such as accountable or not accountable, and optionally additional contextual information such as emotional descriptors or justification. The system can further include data processing and analysis components configured to correlate physiological data with user input to generate behavioral alignment records. These records can be utilized to identify recurring behavioral patterns, physiological triggers, and/or trends over time. The system may further generate behavioral insights and feedback, including quantitative or qualitative indicators of alignment between physiological responses and user-reported behaviors, thereby enabling improved self-awareness and decision-making.

In this manner, the behavioral accountability system of the present invention overcomes long-standing deficiencies in the art by providing a real-time, data-driven mechanism for self-reflection and behavioral alignment. The invention enables users to recognize physiological indicators of emotional or cognitive changes, engage in immediate self-assessment, and improve the consistency between internal states and external behaviors. As a result, the system enhances communication clarity, reduces misunderstandings, and promotes accountability in interpersonal interactions. Furthermore, the invention provides a scalable and adaptable solution that integrates physiological monitoring with behavioral analysis, thereby offering improved transparency, user awareness, and long-term behavioral improvement.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a behavioral accountability system. The system comprises a mobile computing device including one or more processors and a non-transitory memory, and at least one physiological data source configured to collect physiological data of a user, the physiological data including at least heart rate information. The system further includes an accountability software application executed by the one or more processors, wherein the accountability software application is configured to receive physiological data from the physiological data source and establish a baseline physiological profile for the user based on historical physiological data. The application is further configured to analyze incoming physiological data relative to the baseline physiological profile to detect a deviation indicative of a change in a physiological and/or emotional state of the user, generate a trigger event in response to detecting the deviation, and transmit a notification signal to a user interface of the mobile computing device prompting the user to perform a behavioral self-assessment. The application is additionally configured to receive user input corresponding to a behavioral state of the user and store the user input in association with the physiological data to generate a behavioral record.

In another embodiment, a computer-implemented method for facilitating behavioral accountability of a user is disclosed. The method includes receiving physiological data associated with the user, the physiological data including at least heart rate data, and establishing a baseline physiological profile based on historical physiological data. The method further includes monitoring incoming physiological data and detecting a deviation from the baseline physiological profile and generating a trigger event in response to the detected deviation. The method additionally includes transmitting a notification to a user interface prompting the user to perform a self-assessment, receiving input from the user indicative of a behavioral state, and storing the received input together with corresponding physiological data to generate a behavioral alignment record.

In a further embodiment, a method for monitoring physiological data and providing behavioral accountability is provided. The method includes collecting physiological data of a user over time and computing a baseline physiological profile including a baseline heart rate range. The method further includes detecting a heart rate spike based on a comparison of real-time physiological data with the baseline physiological profile and generating and displaying a notification on a mobile device in response to the detected heart rate spike. The method also includes prompting the user to evaluate a current behavioral state and receiving and storing user input representing the behavioral state of the user.

In still another embodiment, a non-transitory computer-readable medium is provided storing instructions that, when executed by one or more processors, cause a computing device to perform operations. The operations include receiving physiological data associated with a user and establishing a baseline physiological profile for the user. The operations further include detecting a deviation in the physiological data relative to the baseline physiological profile and generating a notification signal in response to the deviation. The instructions further cause the computing device to present a user interface prompting a behavioral self-assessment, receive user input corresponding to a behavioral state, and store the user input and associated physiological data to generate behavioral insights.

In some embodiments, the physiological data source comprises a wearable device configured to measure heart rate and transmit the physiological data to the mobile computing device via a wireless communication protocol. The wearable device can include one or more biometric sensors and communication interfaces for continuous or periodic data transmission.

In one embodiment, the accountability software application includes a baseline profiling module configured to adaptively update the baseline physiological profile based on newly received physiological data. The baseline profiling module may continuously refine baseline parameters to account for temporal variations and changes in user-specific physiological conditions.

In one aspect, a graphical representation of physiological data over time on a user interface of the mobile device can include a visual indicator corresponding to the detected heart rate spike and may display temporal trends associated with physiological measurements.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic diagram of exemplary behavioral accountability system of the present invention in accordance with the disclosed architecture;

FIG. 2 illustrates a flow diagram depicting steps for monitoring physiological data of a user and facilitating behavioral accountability based on detected physiological changes, in accordance with the disclosed architecture;

FIG. 3 illustrates a flowchart depicting steps for generating and adaptively updating a personalized physiological baseline profile of a user using the software application in accordance with the disclosed architecture;

FIG. 4 illustrates an exemplary user interface of the mobile computing device configured to present a behavioral accountability notification generated by the accountability software application of the present invention in accordance with one embodiment of the present invention;

FIG. 5 illustrates another exemplary user interface of the mobile computing device configured to present a behavioral accountability check-in interface generated by the accountability software application in accordance with the disclosed architecture; and

FIG. 6 illustrates an exemplary user interface configured to present behavioral insights generated by the accountability software application in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It can be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments can be combined.

As noted above, there exists a long-felt need in the art for an improved system and method for promoting behavioral accountability during interpersonal interactions. There is a need for a solution that enables individuals to take ownership of their words, actions, and behaviors in real time, particularly during conversations that can involve emotional or cognitive stress. Additionally, there exists a need for a system that reduces ambiguity and vagueness in communication, thereby improving transparency and minimizing misunderstandings. Moreover, there is a need for a mechanism that assists individuals in recognizing cognitive biases, emotional reactivity, and illogical thinking patterns, including tunnel vision, that may impair effective communication. Finally, there is a need for a technological solution that provides timely self-assessment prompts and encourages alignment between an individual's internal state and externally expressed behavior.

The present invention, in one exemplary embodiment, is a non-transitory computer-readable medium storing instructions that, when executed by one or more processors, cause a computing device to perform operations. The operations include receiving physiological data associated with a user and establishing a baseline physiological profile for the user. The operations further include detecting a deviation in the physiological data relative to the baseline physiological profile and generating a notification signal in response to the deviation. The instructions further cause the computing device to present a user interface prompting a behavioral self-assessment, receive user input corresponding to a behavioral state, and store the user input and associated physiological data to generate behavioral insights.

Reference will now be made in detail to the present preferred embodiments of the invention, examples of which are illustrated in the accompanying drawings. Wherever possible, the same reference numerals are used in the drawings and the description to refer to the same or like parts.

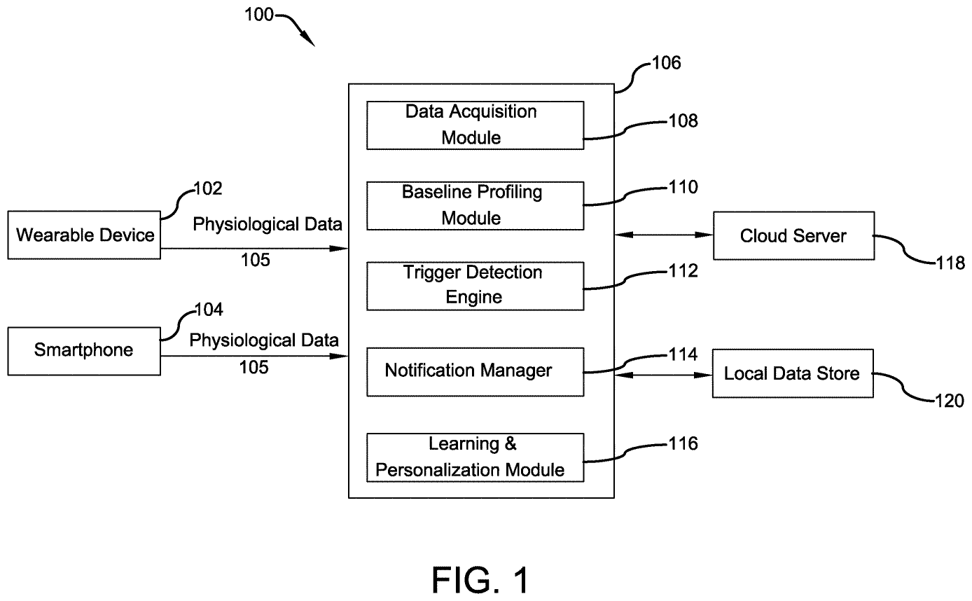

Referring initially to the drawings, FIG. 1 illustrates a schematic diagram of exemplary behavioral accountability system of the present invention in accordance with the disclosed architecture. The behavioral accountability system 100 of the present invention is configured as a smartphone application-based behavioral accountability system to monitor physiological data of a user and facilitate behavioral self-assessment and accountability.

The system 100 includes one or more physiological data sources, including a wearable device 102 configured to collect physiological signals or data types from a user, such as, Electrocardiogram (ECG/EKG), Electroencephalography (EEG), Electromyography (EMG), breathing rate, resting heart rate, present heart rate, body temperature, and heart rate variability (HRV). The smartphone 104 can also include sensors configured to capture physiological or contextual data of the user. The wearable device 102 and/or smartphone 104 are configured to transmit physiological data 105 to the accountability software application 106 via one or more communication interfaces, including wireless communication protocols.

The system 100 further includes an accountability software application 106. In some embodiments, the accountability software application 106 is implemented on or executed by a mobile computing device, such as the smartphone 104, and in some implementations can be fully integrated within the smartphone 104. In other embodiments, the accountability software application 106 can be partially distributed across remote computing resources, such as a cloud server 118. The accountability software application 106 includes a plurality of functional modules implemented as executable instructions stored in memory and executed by one or more processors.

A data acquisition module 108 is configured to receive physiological data 105 from one or more data sources, including the wearable device 102 and/or the smartphone 104. The data acquisition module 108 can include one or more communication interfaces supporting wireless protocols such as Bluetooth®, Wi-Fi, or cellular communication, and is further configured to normalize incoming data streams into a standardized format. In some embodiments, the data acquisition module 108 performs preprocessing operations including filtering, noise reduction, timestamp synchronization, and validation of incoming data. The module supports multi-sensor fusion by aggregating data from multiple physiological sensors, such as heart rate sensors, motion sensors, or temperature sensors.

A baseline profiling module 110 is configured to establish and maintain a personalized baseline physiological profile for the user based on historical physiological data. The baseline profiling module 110 can compute statistical measures including mean, median, variance, and standard deviation of physiological parameters such as resting heart rate, heart rate variability HRV, and activity-adjusted ranges. In some embodiments, the module utilizes adaptive algorithms to continuously update the baseline profile over time, accounting for temporal variations such as circadian rhythms, activity levels, and environmental conditions.

A trigger detection engine 112 is configured to analyze real-time physiological data relative to the established baseline profile and detect deviations indicative of a change in physiological and/or emotional state of the user. The trigger detection engine 112 can implement one or more rule-based logic, threshold comparisons, and machine learning models to identify anomalies, including sudden increases (i.e., changes) in heart rate or deviations in HRV. In the preferred embodiment, the engine 112 applies dynamic thresholds that adapt based on user-specific baseline data and contextual factors, such as motion (i.e., activity level) or time of day. The trigger detection engine 112 is further configured to determine whether a detected deviation satisfies a trigger condition and, upon satisfaction of the trigger condition, generate a trigger event for subsequent processing.

A notification manager 114 is configured to generate and manage notification signals in response to trigger events detected by the trigger detection engine 112. The notification manager 114 can present notifications via a graphical user interface on the smartphone 104, including visual, auditory, or haptic alerts prompting the user to perform a behavioral self-assessment.

A learning and personalization module 116 is configured to analyze user interactions with the accountability software application 106, including responses to notifications and self-assessment inputs, to generate personalized behavioral insights. The learning and personalization module 116 can implement statistical modeling or pattern recognition to identify recurring behavioral patterns and physiological triggers.

The system 100 can further include a cloud server 118 communicatively coupled to the accountability software application 106, wherein the cloud server 118 is configured to store physiological and behavioral data, perform additional processing or analytics, and support synchronization across multiple devices. Additionally, a local data store 120 associated with the smartphone 104 is configured to store physiological data, baseline profiles, and behavioral records locally, thereby enabling offline operation and secure data retention.

It should be noted that the application 106 utilizes physiological data associated with the user, including at least heart rate data obtained from one or more sensors, to detect changes indicative of an emotional, cognitive, and/or physiological state. Upon detecting a deviation from a baseline physiological profile, the application 106 generates a notification signal and presents a prompt to the user via a graphical user interface of the mobile computing device as shown later in the disclosure. The prompt may request the user to assess whether their current behavior is aligned with their internal state, thereby enabling the user to determine whether their thoughts, feelings, and emotions are consistent with their words and actions.

The system 100 along with the application 106 provides clarity and transparency to the user by presenting feedback and insights derived from aggregated physiological and behavioral data. The insights can include patterns of recurring triggers, behavioral tendencies, and alignment metrics, thereby enabling the user to better understand the relationship between physiological responses and behavioral outcomes. The system 100 can also facilitate improved communication by enabling users to reflect on their behavior during interactions with others, thereby reducing misunderstandings and promoting more authentic and accountable communication.

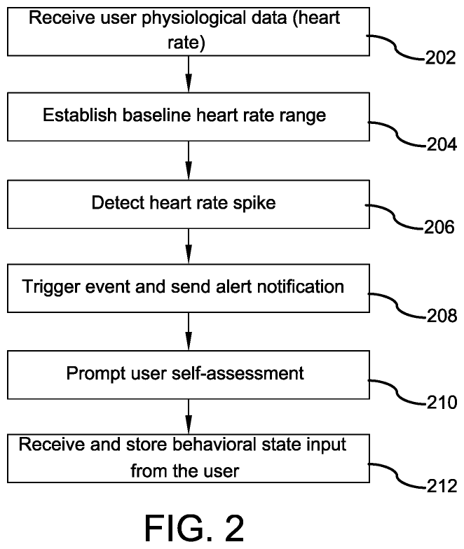

FIG. 2 illustrates a flow diagram depicting steps for monitoring physiological data of a user and facilitating behavioral accountability based on detected physiological changes, in accordance with the disclosed architecture. The method can be performed by the accountability software application 106 described with reference to FIG. 1.

Initially, physiological data associated with a user is received (Step 202). The physiological data can include at least heart rate information and can further include additional biometric signals, such as data types including Electrocardiogram (ECG/EKG), Electroencephalography (EEG), Electromyography (EMG), breathing rate, resting heart rate, present heart rate, body temperature, heart rate variability (HRV), motion data, or other sensor-derived parameters. The physiological data can be obtained from one or more sensors integrated within the wearable device and/or the smartphone and can be received in real time or near real time via one or more communication interfaces.

Then, a baseline physiological profile is established for the user (Step 204). The baseline physiological profile can include at least a baseline heart rate range determined based on historical physiological data collected over a defined time period. In some embodiments, the baseline can be dynamically updated to account for temporal variations, including activity levels and circadian rhythms.

Thereafter, incoming physiological data is analyzed relative to the baseline physiological profile to detect deviations (Step 206). The deviations can include a heart rate spike or anomaly exceeding a predetermined or adaptive threshold. The detection can be performed to identify physiological changes indicative of a shift in emotional, cognitive, and/or physiological state of the user.

At step 208, in response to detecting the deviation, a trigger event is generated. The system then generates and transmits a notification signal to a user interface associated with the smartphone. The notification can include a prompt requesting the user to perform a behavioral accountability check-in, and can be delivered via visual, auditory, or haptic output.

Then, the user is prompted, via an interactive interface, to perform a self-assessment (Step 210). The self-assessment can include evaluating the user's current behavioral, emotional, or cognitive state in response to the detected physiological event. The interface can present selectable options, including classifications such as accountable or not accountable.

Finally, user input corresponding to the behavioral state is received and stored in a data storage medium (i.e., local store 120 of FIG. 1). The stored data can include the selected behavioral classification, associated physiological data of the user at the time of the trigger event, and any additional contextual input provided by the user.

The method of FIG. 2 can be continuously or iteratively performed to provide ongoing monitoring and real-time behavioral accountability support. It should be noted that one or more steps of FIG. 2 can be modified, reordered, or performed in parallel without departing from the scope of the present disclosure.

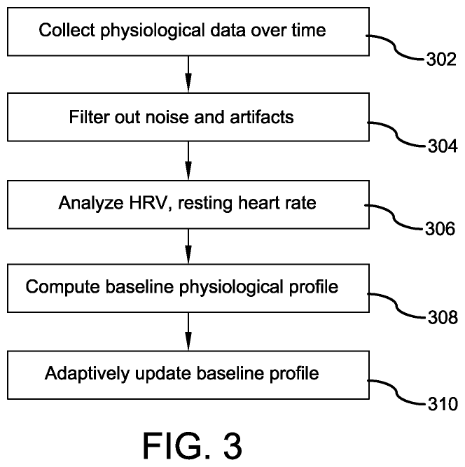

FIG. 3 illustrates a flowchart depicting steps for generating and adaptively updating a personalized physiological baseline profile of a user using the software application in accordance with the disclosed architecture. Initially, physiological data associated with a user is collected over a period of time (Step 302). The physiological data can include data types including Electrocardiogram (ECG/EKG), Electroencephalography (EEG), Electromyography (EMG), breathing rate, resting heart rate, present heart rate, body temperature, heart rate, heart rate variability (HRV), and other biometric signals obtained from one or more sensors of a wearable device and/or the smartphone. The data can be continuously or periodically acquired and can be associated with temporal and contextual metadata, including time of day, user activity, or environmental conditions.

Then, the collected physiological data is processed to filter out noise and artifacts (Step 304). The filtering can include removing erroneous readings, smoothing signal fluctuations, and applying signal processing techniques to improve data quality and reliability. In certain embodiments, adaptive filtering algorithms can be employed.

At step 306, the filtered physiological data is analyzed to derive at least two physiological parameters, including Electrocardiogram (ECG/EKG), Electroencephalography (EEG), Electromyography (EMG), breathing rate, resting heart rate, present heart rate, body temperature, heart rate variability (HRV), resting heart rate, present heart rate, and other statistical or derived metrics. The analysis can include calculating statistical measures such as mean values, standard deviation, variance, and temporal patterns.

Thereafter, a baseline physiological profile is computed based on the analyzed physiological data (Step 308). The baseline physiological profile can include at least a baseline heart rate range, expected variability thresholds, and other personalized physiological characteristics representative of the user under normal conditions. In the preferred embodiment, the baseline profile comprises different baseline states corresponding to different user conditions, such as resting, active, or stressed states.

Finally, the baseline physiological profile of the user is automatically adaptively updated over time (Step 310). The adaptive updating can include incorporating newly collected physiological data, adjusting baseline parameters based on long-term trends, and refining thresholds to reflect changes in physiological condition of the user.

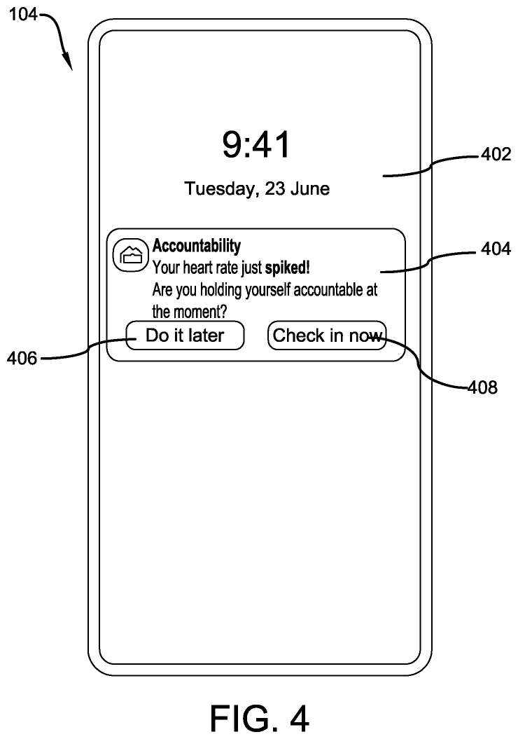

FIG. 4 illustrates an exemplary user interface of the mobile computing device configured to present a behavioral accountability notification generated by the accountability software application of the present invention in accordance with one embodiment of the present invention. The user interface can be displayed on the smartphone 104 and is configured to provide real-time prompts to a user in response to detected physiological trigger events, as described with reference to FIGS. 1-3.

As shown, a notification panel 404 is displayed on the interface 402, the notification panel 404 being generated by the notification manager 114 (FIG. 1) in response to a detected physiological deviation, such as a heart rate spike. The notification panel 404 can include a message indicating that physiological state of the user has changed and prompts the user to evaluate their current behavior, for example by asking whether the user is holding themselves accountable.

The notification panel 404 further includes one or more interactive user interface elements configured to receive user input. In the illustrated embodiment, a first selectable input element 406 is provided, corresponding to an option to defer or dismiss the accountability prompt (i.e., “Do it later”). A second selectable input element 408 is provided, corresponding to an option to immediately respond to the accountability prompt (i.e., “Check-in now”).

In various embodiments, the user interface 402 may further display additional notifications or system messages concurrently with the accountability notification, illustrating that the accountability prompt can be integrated within a broader notification environment of the smartphone 104.

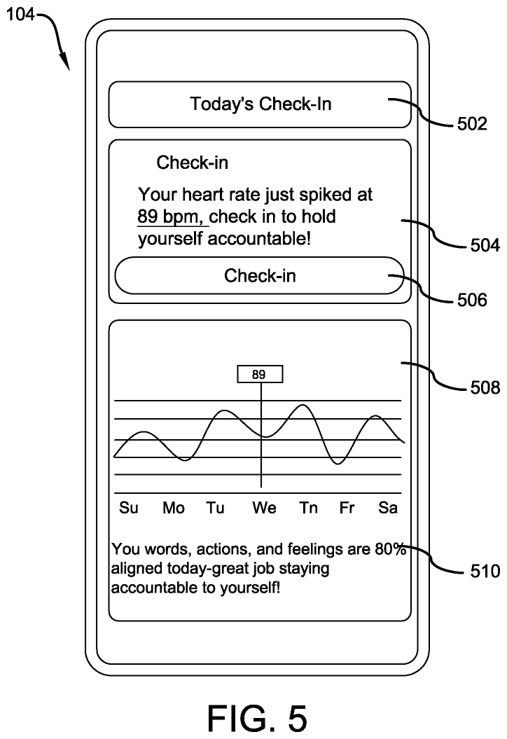

FIG. 5 illustrates another exemplary user interface of the mobile computing device configured to present a behavioral accountability check-in interface generated by the accountability software application in accordance with the disclosed architecture. As shown, the user interface 502 presents a check-in interface associated with a detected physiological trigger event. The user interface 502 includes a check-in notification panel 504 indicating that physiological data, such as heart rate, of the user has exceeded a threshold (such as a detected spike), and prompting the user to engage in a behavioral accountability check-in. The check-in notification panel 504 can include contextual information, such as the measured heart rate value, and may provide explanatory text encouraging the user to reflect on their current behavioral and emotional state.

An interactive input element 506, such as a selectable button is configured to initiate or confirm the user's check-in response. Selection of the input element 506 may cause the accountability software application 106 to record behavioral state and present additional input options for further self-assessment.

The user interface 502 additionally includes a physiological data visualization panel 508 configured to present a graphical representation of the user's physiological data over time. In the illustrated embodiment, the physiological data visualization panel 508 includes a graph illustrating heart rate measurements across a defined time interval, with a marker indicating a detected deviation or spike event. The graphical representation can include axes, labels, and indicators corresponding to temporal data points and physiological values.

A feedback and insight section 510 is further presented within the user interface 502, providing a summary of behavioral alignment based on the user's inputs and associated physiological data. The feedback can include a quantitative or qualitative assessment, such as a percentage alignment between words, actions, of the user and emotions, and can include encouraging or corrective messaging.

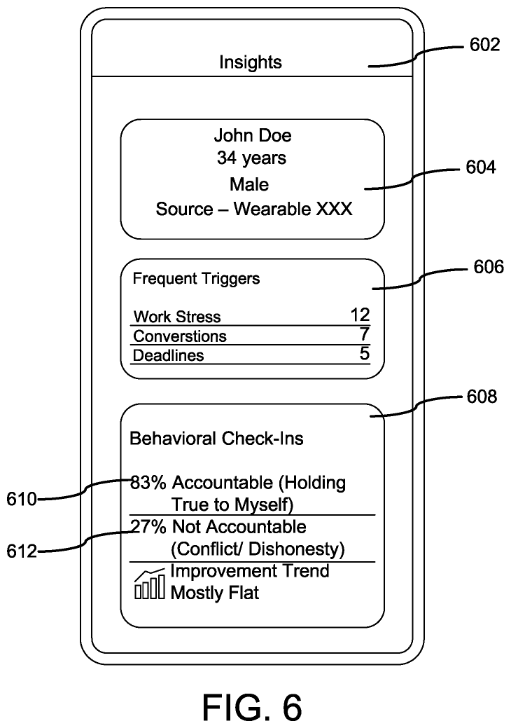

FIG. 6 illustrates an exemplary user interface configured to present behavioral insights generated by the accountability software application in accordance with one embodiment of the present invention. The user interface 602 can be displayed on a mobile computing device, such as the smartphone 104, and is configured to provide aggregated and analyzed information based on previously collected physiological data and user behavioral inputs.

As shown, the user interface 602 includes a plurality of display regions presenting different categories of user-related information. A user profile section 604 is provided for displaying identifying and contextual information associated with the user, such as name, age, and data source information corresponding to a wearable device. The user profile section 604 can be configured to associate physiological and behavioral data with a specific user identity.

The user interface 602 further includes a trigger analysis section 606 configured to present one or more frequently occurring physiological or contextual triggers identified by the accountability software application 106. The trigger analysis section 606 can include a list of trigger categories, such as work-related stress, conversations, or deadlines, along with corresponding occurrence counts or frequency indicators. These triggers can be determined based on correlations between physiological deviations and contextual data over a defined period.

A behavioral check-in summary section 608 is also provided, configured to present aggregated results of user self-assessments. The behavioral check-in summary section 608 can include quantitative metrics indicating the proportion of accountable versus non-accountable user responses, as well as descriptive labels associated with each category. In the illustrated embodiment, the behavioral check-in summary section 608 includes a first metric 610 representing a percentage of accountable behavior and a second metric 612 representing a percentage of non-accountable behavior, such as behavior associated with conflict or dishonesty.

The user interface 602 can further include a trend visualization element indicating changes in user behavior over time, such as an improvement trend graph or summary descriptor. Additionally, the interface can include selectable elements enabling the user to access more detailed reports or extended historical data, such as data corresponding to a defined time period (i.e., last 30 days).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “behavioral accountability system”, “physiological signals monitoring and behavioral accountability system”, “physiological data and real-time user self-assessment system”, and “system” are interchangeable and refer to the physiological signals monitoring and behavioral accountability system and method 100 of the present invention.

Notwithstanding the forgoing, the physiological signals monitoring and behavioral accountability system and method 100 of the present invention can be of any suitable configuration as is known in the art without affecting the overall concept of the invention, provided that it accomplishes the above stated objectives. One of ordinary skill in the art will appreciate that the physiological signals monitoring and behavioral accountability system and method 100 shown in the FIGS. are for illustrative purposes only, and that many other configurations of the physiological signals monitoring and behavioral accountability system and method 100 are well within the scope of the present disclosure. Although the dimensions of the physiological signals monitoring and behavioral accountability system and method 100 are important design parameters for user convenience, the physiological signals monitoring and behavioral accountability system and method 100 can be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A behavioral accountability and monitoring system comprising:
a mobile computing device;
an accountability application;
a wearable device;
a plurality of physiological data types;
a communication interface;
a data acquisition module; and
a baseline profiling module;
wherein said wearable device is configured to collect said plurality of physiological data types from a user of said wearable device;
wherein said plurality of physiological data types selected from at least two of the group consisting of Electrocardiogram (ECG/EKG), Electroencephalography (EEG), Electromyography (EMG), breathing rate, resting heart rate, present heart rate, body temperature, and heart rate variability (HRV);
wherein said wearable device and said mobile computing device comprise multiple physiological sensors configured to capture said plurality of physiological data types of the user;
wherein at least one of said wearable device and said mobile computing device are configured to transmit said plurality of physiological data types to said accountability application with at least one said communication interface;
wherein said data acquisition module is configured to receive said plurality of physiological data types from at least one of said wearable device and said mobile computing device; and
wherein said baseline profiling module is configured to establish and maintain a personalized baseline physiological profile for the user based on historical said plurality of physiological data types.

2. The behavioral accountability and monitoring system of claim 1, wherein said data acquisition module performs at least two preprocessing operations selected from the group consisting of filtering, noise reduction, timestamp synchronization, and validation of incoming data.

3. The behavioral accountability and monitoring system of claim 2, wherein said data acquisition module aggregates data from said multiple physiological sensors selected from the group consisting of heart rate sensors, motion sensors, and temperature sensors.

4. The behavioral accountability and monitoring system of claim 3, wherein said baseline profiling module computes statistical measures selected from the group consisting of mean, median, variance, and standard deviation of said plurality of physiological data types.

5. The behavioral accountability and monitoring system of claim 4 further comprising a trigger detection engine, wherein said trigger detection engine is configured to analyze real-time said plurality of physiological data types relative to said personalized baseline physiological profile to determine said statistical measures.

6. The behavioral accountability and monitoring system of claim 5 further comprising a trigger detection engine, wherein said trigger detection engine is configured to analyze real-time said plurality of physiological data types relative to said personalized baseline physiological profile to determine trigger deviations indicative of a change in physiological state of the user.

7. The behavioral accountability and monitoring system of claim 6, wherein said trigger deviations apply dynamic thresholds that adapts said personalized baseline physiological profile based on user-specific baseline data and contextual factors selected from the group consisting of activity level and time of day.

8. The behavioral accountability and monitoring system of claim 7 further comprising a notification manager configured to generate and manage notification signals in response to said trigger deviations detected by said trigger detection engine.

9. The behavioral accountability and monitoring system of claim 8, wherein said notification manager presents notifications with a graphical user interface on said mobile computing device, and wherein said notifications are selected from one or more of group consisting of a visual alert, an auditory alert, and a haptic alert.

10. The behavioral accountability and monitoring system of claim 9 further comprising a personalization module configured to analyze user interactions with said accountability application comprising responses to notifications and self-assessment inputs for generating said personalized baseline physiological profile.

11. A behavioral accountability and monitoring system comprising:
a mobile computing device;
an accountability application;
a wearable device;
a plurality of physiological data types;
a communication interface;
a data acquisition module;
a baseline profiling module; and
a trigger detection engine;
wherein said wearable device is configured to collect said plurality of physiological data types from a user of said wearable device;
wherein said plurality of physiological data types selected from at least two of the group consisting of Electrocardiogram (ECG/EKG), Electroencephalography (EEG), Electromyography (EMG), breathing rate, resting heart rate, present heart rate, body temperature, and heart rate variability (HRV);
wherein said wearable device and said mobile computing device comprise multiple physiological sensors configured to capture said plurality of physiological data types of the user;
wherein at least one of said wearable device and said mobile computing device are configured to transmit said plurality of physiological data types to said accountability application with at least one said communication interface;
wherein said data acquisition module is configured to receive said plurality of physiological data types from at least one of said wearable device and said mobile computing device;
wherein said baseline profiling module is configured to establish and maintain a personalized baseline physiological profile for the user based on historical said plurality of physiological data types; and
wherein said trigger detection engine is configured to analyze real-time said plurality of physiological data types relative to said personalized baseline physiological profile to determine statistical measures.

12. The behavioral accountability and monitoring system of claim 11, wherein said data acquisition module performs at least two preprocessing operations selected from the group consisting of filtering, noise reduction, timestamp synchronization, and validation of incoming data.

13. The behavioral accountability and monitoring system of claim 11, wherein said data acquisition module aggregates data from said multiple physiological sensors selected from the group consisting of heart rate sensors, motion sensors, and temperature sensors.

14. The behavioral accountability and monitoring system of claim 11, wherein said baseline profiling module computes said statistical measures selected from the group consisting of mean, median, variance, and standard deviation of said plurality of physiological data types.

15. The behavioral accountability and monitoring system of claim 11, wherein said trigger detection engine is configured to analyze real-time said plurality of physiological data types relative to said personalized baseline physiological profile to determine trigger deviations indicative of a change in physiological state of the user.

16. The behavioral accountability and monitoring system of claim 15, wherein said trigger deviations apply dynamic thresholds that adapts said personalized baseline physiological profile based on user-specific baseline data and contextual factors selected from the group consisting of activity level and time of day.

17. A method of behavioral accountability and monitoring, the method comprising the steps of:
providing a mobile computing device, an accountability application, a wearable device, a plurality of physiological data types, a communication interface, a data acquisition module, a baseline profiling module, and a trigger detection engine;
collecting said plurality of physiological data types from a user wearing said wearable device;
wherein said plurality of physiological data types selected from at least two of the group consisting of Electrocardiogram (ECG/EKG), Electroencephalography (EEG), Electromyography (EMG), breathing rate, resting heart rate, present heart rate, body temperature, and heart rate variability (HRV);
wherein said wearable device and said mobile computing device comprise multiple physiological sensors configured to capture said plurality of physiological data types of the user;
wherein at least one of said wearable device and said mobile computing device are configured to transmit said plurality of physiological data types to said accountability application with at least one said communication interface;
receiving with said data acquisition module said plurality of physiological data types from at least one of said wearable device and said mobile computing device;
establishing and maintaining with said baseline profiling module a personalized baseline physiological profile for the user based on historical said plurality of physiological data types; and
analyzing in real-time with said trigger detection engine said plurality of physiological data types relative to said personalized baseline physiological profile to determine statistical measures.

18. The method of behavioral accountability and monitoring of claim 17, wherein said trigger detection engine is configured to analyze real-time said plurality of physiological data types relative to said personalized baseline physiological profile to determine trigger deviations indicative of a change in physiological state of the user.

19. The method of behavioral accountability and monitoring of claim 18, wherein said trigger deviations apply dynamic thresholds that adapts said personalized baseline physiological profile based on user-specific baseline data and contextual factors selected from the group consisting of activity level and time of day.

20. The method of behavioral accountability and monitoring of claim 17 further comprising a personalization module configured to analyze user interactions with said accountability application comprising responses to notifications and self-assessment inputs for generating said personalized baseline physiological profile.
